(19) United States
(12) Patent Application Publication
(10) Pub. No.: US 2007/0219626 A1
(43) Pub. Date: Sep. 20, 2007

(54) Stents made of biodegradable and non-biodegradable materials

(76) Inventors: Giovanni Rolando, Chivasso (IT); Maria Curcio, Saluggia (IT); Andrea Grignani, Chieri (IT); Paolo Gaschino, Castagneto Po (IT)

Correspondence Address: POPOVICH, WILES & O'CONNELL, PA;650 THIRD AVENUE SOUTH, SUITE 600, MINNEAPOLIS, MN, 55402, US

(21) Appl. No.: 11/717,797

(22) Filed: Mar. 13, 2007

(30) Foreign Application Priority Data
Mar. 16, 2006 (EP) 06425174.7

(51) Int. Cl. A61F 2/90

(52) U.S. Cl. 623/1.16; 623/1.38; 623/1.42

(57) ABSTRACT

A stent comprising a plurality of annular elements aligned in the longitudinal direction of extension of the stent and selectively expandable between a radially-contracted condition and a radially-expanded condition as well as a series of connecting elements that extend in the longitudinal direction of extension of the stent to connect the annular elements. The annular elements and the connecting elements are made, respectively, of non-biodegradable material and of biodegradable material. The structure of the stent thus comprises a part of non-biodegradable material, destined to remain long-term at the site of implantation, and a part of biodegradable material, destined to disappear within a longer or shorter period after implantation.

The application from which this application claims foreign priority, European Patent Application No. 06425174.7, filed Mar. 16, 2006, is hereby incorporated by reference.

### FIELD OF THE INVENTION

The invention relates to stents. This term in general indicates expandable endoprostheses capable of being implanted into a lumen in a human or animal body, such as for example a blood vessel, to reestablish and/or maintain its patency.

Stents usually take the form of tubular devices that operate to maintain a segment of the blood vessel or other anatomical lumen open. Over recent years, stents have become established for use to treat stenoses of arterioschlerotic nature in blood vessels such as the coronary arteries. The field of application is now gradually extending to other districts and regions of the body, including the peripheral regions.

### BACKGROUND OF THE INVENTION

The scientific and technical literature concerning stents, including that concerning patents, is very extensive. EP-A-0 806 190, EP-A-0 850 604, EP-A-0 875 215, EP-A-0 895 759, EP-A-0 895 760, EP-A-1 080 738, EP-A-1 088 528, EP-A-1 103 234, EP-A-1 174 098, EP-A-1 212 986, EP-A-1 277 449, EP-A-1 449 546, as well as European Patent Application No. 05001267.3, are related documents assigned to the present Assignee.

In this field, a line of research has aimed specifically at producing biodegradable stents (for example of bioerodible or bioabsorbable material). In other words, these are stents made of materials (for example using polymers but also metals or alloys) such that, after implantation of the stent, they undergo degradation that in practice causes the disappearance of the stent. Examples of this line of research include EP-A-0 554 082 and EP-A-0 894 505.

The development of biodegradable stents takes as its starting point the following consideration: it is known that, after implantation of a stent, the risk that the treated vessel undergoes restenosis, if this is to occur, exists in the first 6 to 12 months. Whereas the risk of this happening in the longer term is very small indeed. From the biological standpoint the explanation, as far as is known at present, is that restenosis is caused by a series of factors linked chronologically to implantation of the stent. If during the time span indicated these factors are overcome, this means that the lesion of the blood vessel has healed and, in practical terms, there is no longer any need to have a stent present that maintains patency. Thus a stent that, having completed its function, disappears from the treated blood vessel and eliminates the presence of a foreign body would be desirable.

Apart from the conceptual interest, now studied for many years, the most evident obstacle to be overcome in producing a stent of biodegradable material lies in the fact that in order to have adequate radial strength comparable to that of traditional stents the structure must be of a thickness that compromises its basic functional aspects (ease of implantation, etc.) and that causes problems of safety (risk of thrombosis due to turbulence). Furthermore, biodegradable materials such as bioerodible polymers are in general known to cause inflammatory conditions, which are the harbinger of restenosis. To implant such a mass of these polymers as is needed to guarantee the required initial strength may lead to serious problems of biocompatibility.

Biodegradable stents of a metallic type (based on corrosible metals, such as for example magnesium) are less widespread. Indeed, the scientific community has up to now been concerned about having a rapid and massive local release of metal ions resulting from corrosion, about the true predictability of the time span during which the mechanical strength is lost, and about the progression of the phenomenon.

Independent of all other considerations (choice of materials, kinetics of erosion or absorption, etc.) stents of the biodegradable type must come to terms with a basic problem. Before it is fully biodegraded, the stent (or better what remains of the stent as it undergoes gradual degradation) constitutes a sort of “remnant” that can undergo deformation or even dislocation from the site of implantation. These phenomena may be dangerous because they might cause occlusion of the treated blood vessel or might trigger the formation of thrombi.

Research concerning stents has gradually widened to include other details of production, and in particular to drug eluting stents (DES). This field deals with the possibility of applying onto the stent, or otherwise associating to the stent, substances having the nature of a drug. These substances are thus capable of exercising specific activity at the stent implantation site. In particular drugs with action antagonistic to restenosis have been associated with the stent.

For example, EP-A-0 850 604 describes the possibility of providing stents with sculpturing comprising, for example, cavities capable of receiving one or more drugs useful for the prevention or treatment of restenosis and/or substances appropriate for correct use of the stent (adhesion, release modalities, kinetics, etc.). This surface sculpturing is characterized both by the shape and surface area of the cavity, and by its in-depth profile. For example, the cavities may be cavities with circular openings or oval-shaped openings or again elongated openings. Alternatively, they may take the form of an appropriate alternation of cavities with openings of different types depending on the release requirements. The in-depth profile may be “U” or “V” shaped, or again in the form of a vessel with or without a superficial part entirely dedicated to receiving the substances of interest indicated above. This superficial part may have the aspect of a sort of continuous layer only on the outer surface of the stent.

A great deal of work has been dedicated over recent years to identifying the nature of the material, and in particular of the drug, loaded onto the stent. This may consist of a single drug, a pair of drugs, or a series of drugs with similar, synergistic or diversified action. Alongside pharmacologically-active molecules, the stent may also carry substances functioning as adjuvants to the pharmacologically-active substances, such as polymers or excipients of various types. The function may be to stabilize the active principle or principles, or may be directed to regulating release kinetics (slowing or accelerating release). The polymers/excipients may be mixed with the drug or drugs, or may be in separate layers with respect to the pharmacologically-active substances. For example, the polymers/excipients may form a sort of stopper of biodegradable polymer over the hollow or alternatively create a stratified structure with successive layers of drug and polymer.

Although this type of application is not at present considered particularly attractive among the scientific community, radioactive substances may be loaded onto the stent.

Also in regard to these aspects, the technical and scientific literature and that concerning patents is very extensive, as is shown, as well as by some of the documents already quoted, by others such as for example, EP-A-0 551 182, EP-A-0 747 069, EP-A-0 950 386, EP-A-0 970 711, EP-A-1 254 673, EP-A-1 254 674, WO-A-01/87368, WO-A-02/26280, WO-A-02/26281, WO-A-02/47739, WO-A-02/056790 and again WO-A-02/065947 as well as the literature quoted in these documents. These documents and literature do not in any way exhaust the body of literature on the subject.

With regard to the choice of drug with functions antagonistic to restenosis, drugs known as rapamycin (sirolimus) and FK506 (tacrolimus) have taken on particular importance.

The problems connected to the use of drugs on the stent are not, however, limited to the choice of drug alone (the identification of the substance or substances used) but also involve several further aspects. These further aspects include: (1) the physical form of the substance to be loaded; (2) the loading technique of the material; (3) the technique for cleaning off excess material deposited; and (4) stabilization of the material.

The loading techniques must take into account the nature (that is the physical form) of the substance or substances to be loaded onto the stent. Some loading techniques of known type essentially operate in an indirect fashion, since they substantially entail applying a coating onto the stent, typically of polymeric material (for example polymers of methacrylate, polyurethane, polytetrafluoroethylene (PTFE), hydrogel or mixtures of hydrogel/polyurethane, especially PTFE) to or in which the drug to be applied onto the stent is bonded and/or dissolved before application of the coating. The coating is then stabilized by polymerization.

Other techniques substantially entail starting from agents in liquid form or from solutions or dispersions with low viscosity. In most cases considered the drugs of interest are substances that, originally, or in the form in which they are available in commerce, are in the form of powders (with different granulometry). The simplest solution entails loading the stent by immersing it in a vector, typically a liquid, in which is dissolved, suspended or in any case present the substance or substances to be loaded onto the stent. This technique, which may also if necessary be done under vacuum, is known in the art as dipping.

For example, a solution is described in the document WO-A-02/065947 in which the stent is brought into contact with a solution of FK506 in an aqueous or organic solvent (typically in alcohol, such as ethanol, at a concentration of 3.3 mg of FK506 in 1 ml of ethanol). This, for example, comes about through dripping, spraying or immersing, preferably under vacuum. The stent is then dried, preferably until the solvent is eliminated, and the operation is repeated from 1 to 5 times. Subsequently the stent is, if necessary, washed once or more than once with water or isotonic saline solution, and finally is dried.

To complete the overview of the background of the present invention, it must be mentioned that from the first developments of stent technology (see for example EP-A-0 540 290) it has been very clear to technicians that the characteristics of longitudinal flexibility of a stent come into play in two different contexts: (1) when the stent, arranged in its radially-contracted condition on the implantation catheter, is advanced through the patient's vascular system until it reaches the implantation site (so-called “trackability”), and (2) when the stent, implanted in its radially-expanded condition at the treatment site and after the implantation catheter has been removed, must correctly maintain its implanted position at a vascular site subject to cyclic deformation under the action of the pulsating blood flow and/or that of the cardiac mass that contracts rhythmically, without altering the natural compliance of the blood vessel.

### SUMMARY OF THE INVENTION

The invention aims to take into account a series of essential factors that have to date been linked in a more or less indissoluble fashion to the production of stents of the drug eluting type, and that is: (1) the complexity of the operation of loading the drug or active principle; (2) the need, where a coating is produced on the stent, in which the drug to be applied to the stent is bonded and/or dissolved, to take into account the characteristics of the coating, and the possible subsequent elimination of the coating itself; (3) the difficulty of achieving selective coatings, that is coatings limited to circumscribed areas of the stent; (4) the objective difficulty of loading a plurality of different agents with a limited number of stages; and (5) the critical aspect intrinsically linked to the contemporary loading of more than one agent and if necessary excipients or other substances that can contribute to controlling release kinetics.

The invention provides a solution that is able to overcome the above difficulties in a radical fashion.

The present invention provides a stent having the characteristics indicated specifically in the attached claims. The claims form an integral part of the disclosure provided here in regard to the invention.

The invention is based on the concept of stents made of biodegradable material (for example, bioerodible or bioabsorbable), that is a material that, when exposed to the biological environment in which the stent is implanted (typically a vascular site), undergoes a phenomenon of decay that brings about its gradual disappearance. For the purposes of the present application, the definition of biodegradable material thus leaves completely out of consideration the mechanism (erosion, absorption, corrosion, etc.) that underlies this behavior.

The solution described here thus concerns, in the presently preferred embodiment, a stent comprising a tubular structure that is selectively expandable between a radially-contracted condition, in which the stent is capable of being carried to the site of implantation, and a radially-expanded condition, in which the stent, positioned at the implantation site, is able to sustain the blood vessel subjected to treatment in an open, patent position, thus eliminating the stenosis, said tubular structure comprises a part of non-biodegradable material and a part of biodegradable material.

In the presently preferred embodiment, the solution described here substantially entails developing what might be called a hybrid stent, comprising a basic structural part and a part made of biodegradable material. The basic structural part is made of non-biodegradable material and thus is destined to remain at the implant site (thus providing the supporting action to the walls of the treated blood vessel without having a negative effect on the natural feature of compliance of the blood vessel). This basic structural part typically is comprised of a small number of expandable annular elements, connected together or otherwise, that provide the principal radial supporting function. The part made of biodegradable material is destined to provide, together with the basic structural part, structural coherency and flexibility to the stent when it is implanted. The part made of biodegradable material cooperates in the supporting function (for example local support of the plaque, avoiding prolapse) but is destined to disappear some months after implantation, once healing of the treated blood vessel has been achieved.

The solution described here offers a significant contribution to the field of medicated stents. The part of the stent made of biodegradable material represents an excellent drug carrier, from which the drugs can be released slowly over time and, given the masses involved, one that can be loaded with much greater quantities than the devices in current use.

As will be better understood in the detailed description of some exemplary embodiments that follows, the solution described here makes it possible to greatly simplify the operation of loading drug or active principle, making the choice of other components (vectors, excipients, etc.) associated to the drug much less critical. Furthermore, drug loading of the selective type can more easily be achieved (that is loading limited to circumscribed areas of the stent). The possible use of a plurality of different agents, the contemporary loading of more than one agent, or if required excipients or other substances capable of contributing to the control of release kinetics can more easily be achieved.

It will also be understood that the solution described here overcomes the typical demonstrated drawbacks of stents of the biodegradable type. The part of the stent that is biodegradable no longer is required to be massive, but can be of dimensions compatible with those of stents in current use. Once the biodegradable part has disappeared, the non-biodegradable basic part of the stent, of itself minimally invasive, remains solidly and precisely on site.

### BRIEF DESCRIPTION OF THE DRAWINGS

The invention will now be described, by way of non-limiting examples, with reference to the attached drawings.

FIGS. 1 and 2 show, in diagram form, the basic part of the stent described here.

FIGS. 3, 4, and 5 correspond to three possible embodiments of the stent described here.

### BRIEF DESCRIPTION OF THE PREFERRED EMBODIMENTS

In general, the solution according to the invention lends itself to being produced within the sphere of a stent structure of the type described, for example, in EP-A-0 875 215, comprising: (1) a plurality of annular elements the walls of which follow a looped path (typically sinusoidal or approximately sinusoidal) aligned along the axis of the stent (direction z in the figures) and selectively expandable between a radially-contracted position and a radially-expanded position to achieve the expansion movement of the stent, and (2) a network of longitudinal connecting elements (in general known as “links”) that extend like a bridge to connect the annular elements; said connecting elements are in general capable of extending and contracting in the longitudinal direction of the stent (for example by effect of a general λ or Ω conformation, see in this connection EP-A-0 875 215) in order to give the stent the properties of longitudinal flexibility required to guarantee that it displays the appropriate “trackability” during its implantation.

In other words, these are stents in which the dual functions of radial expandability and longitudinal flexibility are, in distinct and separate fashion, provided by two different sets of members, that is the annular elements with looped wall (radial expandability of the stent) and the connecting elements or links (longitudinal flexibility).

From the conceptual standpoint, the solution described here is based on recognition of the fact that the presence of both parts or components of the stent are required, from the structural standpoint, only during the phase of stent implantation (inserting the stent and guiding it towards the implant site employing a catheter, expansion of the stent at the implant site). The network of longitudinal connecting elements or links concludes its function during implantation and the immediately subsequent phases (for example, to provide a supporting action of the plaque, avoiding its prolapse inside the treated vessel). Having completed its function and obtained healing of the treated site, the connecting structure may in fact disappear. Hence the choice, adopted in the solution described here, of producing this part, at least to a substantial extent, of biodegradable material. That is of material destined to disappear during a shorter or longer timeframe (once again it is mentioned that the term “biodegradable” is used here in its widest sense, without specific reference to any mechanism underlying the gradual disappearance of the material itself).

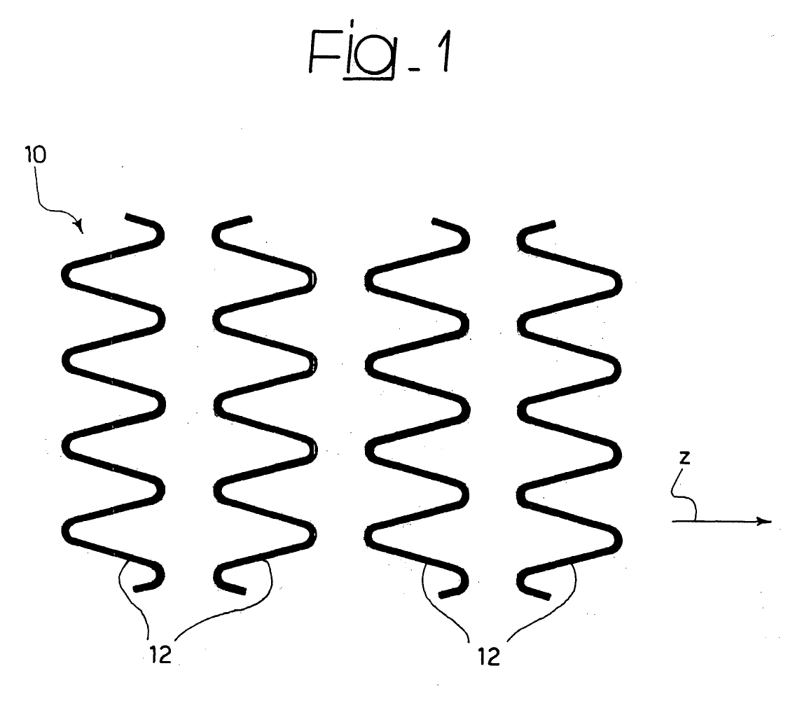

FIG. 1 shows (in ideal flat development, following the practice generally adopted to represent the structure of stents) the basic part of the stent of the type described here. In the specific case, this basic part, indicated with 10, is comprised of four annular elements 12 that, with the stent considered in its typical tubular configuration, present an overall cylindrical shape and a looped path. In the specific case, the looped path in question is represented by a sinusoidal trajectory and the various elements 12 are positioned mutually such that their sine waves are in phase opposition. In other words, following FIG. 1 from left to right, note that, for example, the first and second elements 12 present opposed valleys and peaks (where the first element 12 presents a valley facing towards the second element 12, the second element 12 presents a valley facing towards the first, and so on). In a similar fashion, the second and third elements 12 present opposed valleys and peaks, while the same is also true of the third and fourth elements 12. The stent according to the invention is in general capable of containing any plural number of elements 12.

From the theoretical standpoint, each of the elements 12 might be seen as representing a stent even when taken singly: nevertheless, the solution described here relates to stents in which a plurality of these elements are present, connected one to another by links or longitudinal connecting elements, whose characteristics will be better described below.

In the solution described here, the part of the structure of the stent comprising the elements 12 is made of a “non-biodegradable” material, that is of durable material of the type normally used to make stents and in general materials are indicated such as stainless steel, cobalt-chromium alloy, etc., if appropriately surface-treated by applying a layer of biocompatible carbonaceous material in the way described, for example, in U.S. Pat. No. 4,624,822, U.S. Pat. No. 4,758,151, U.S. Pat. No. 5,084,151, U.S. Pat. No. 5,133,845, U.S. Pat. No. 5,370,684, U.S. Pat. No. 5,387,247, and U.S. Pat. No. 5,423,886.

The term “durable” is thus used in opposition to the term “biodegradable”. In substance, the basic part indicated with 10 constitutes, in the solution described here, the part of the stent destined to remain at the implant site in the long term, which is after the parts made of biodegradable material have disappeared.

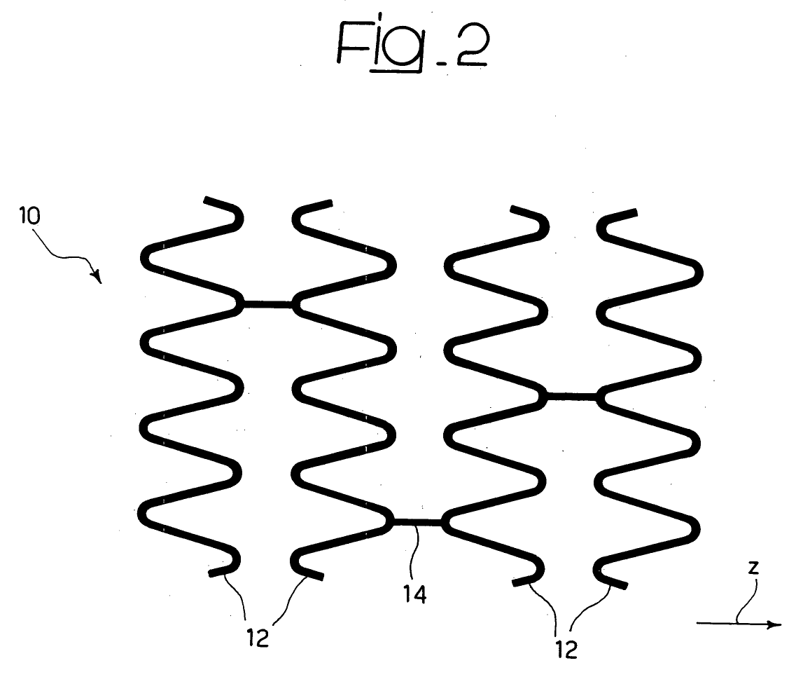

FIG. 2 shows that, in some possible embodiments of the solution described here, longitudinal connecting elements 14 may be situated between the elements 12 and may extend in the fashion of a bridge between the elements 12 in the longitudinal direction of the stent, indicated as reference z.

Independently of their extension in the longitudinal sense, axial with regard to the stent, the elements 14 must not be confused with the connecting elements or “links” (indicated on the contrary with 18) destined to give the stent overall the characteristics of longitudinal flexibility. This is because: (1) the elements 14 are typically made in the form of linear elements (“struts”) of fixed length and, as such, are non-extensible. As such, they may not therefore co-operate in any way, in a stent of the type described here, to provide the longitudinal flexibility, which indeed presupposes the fact that the connecting elements or links may vary in length; and (2) in any case the elements 14, even if extensible, are present in a limited number (for example one or two elements 14 placed to connect two adjacent elements 12) and in consequence they only comprise a minor part (less than 50%) and usually a very minor part (no more than 25%, usually less than 20% or less than 10%) of the overall number of elements that extend in the longitudinal direction (z axis) of the stent to link adjacent elements 12.

If present, the elements 14 are usually made of the same material as the elements 12, and usually made as a piece with the elements 12 in the sphere of processing procedures (laser cutting of a micro-tube or hypotube) normally used to manufacture stents in current use.

In substance, if present, the elements 14 perform the sole function of preventing the individual elements 12 of the basic structure 10, destined to remain in place long-term after implantation of the stent, from undergoing the undesired phenomena of reciprocal displacement and/or taking on an undesired orientation. In other words, the elements 14 essentially act as spacers.

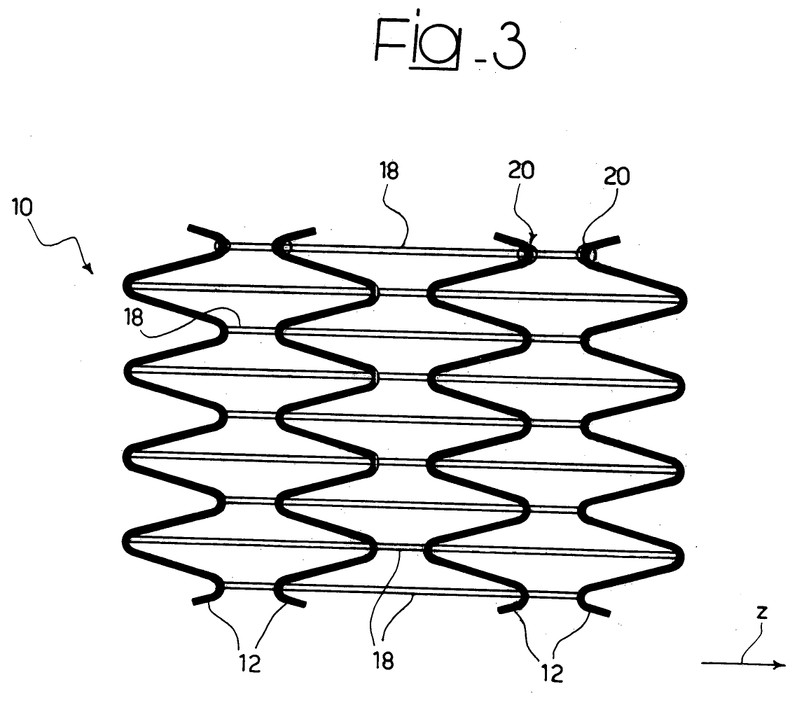

FIGS. 3 to 5 illustrate some ways of coupling to a basic structure 10 illustrated in FIG. 1 a set of connecting elements 18 destined to complete the structure of the stent so as to give the stent the features of mechanical coherency necessary for the implantation stage.

In particular, in the embodiment in FIG. 3, the connecting elements 18 are comprised of linear bodies (in practice fibers or “spaghetti”) of biodegradable polymeric material, preferably associated (in a manner described more clearly below) to at least one active principle such as for example an agent antagonistic to restenosis.

The elements 18 are based on a biodegradable material that in general presents characteristics of elasticity, that is longitudinal extensibility. This means that the stent formed of the series of elements 12 and elements 18 is capable of flexing longitudinally along its z axis so as to be able to follow the tortuous path within the treated patient's vascular system along which it advances towards the implant site.

For example, a polymer material presenting the required characteristics may be selected from among: polylactic acid; poly-ε-caprolactone; polyorthoesters; polyanhydrides; poly-3-hydroxybutyrate; polyaminoacids such as polyglycine; polyphosphazenes; polyvinyl alcohol; low molecular weight polyacrylates; and copolymers of these.

Among metallic biodegradable materials, iron and magnesium may be used.

These materials lend themselves to being produced in the form of filiform elements such as fibers or spaghetti with a circular section presenting a diameter on the order of 0.1 mm.

Their physical characteristics guarantee that the elements 18 will contribute in full to providing the necessary characteristics of mechanical coherency of the stent without undergoing undesired fracture. At the same time, the material of the type described is able to ensure complete biodegradation (thus in practice the disappearance of the elements 18) within a period of time on the order of one to six months after implantation of the stent.

The biodegradable material of the elements 18 lends itself to being loaded with an active principle such as, for example, an agent antagonistic to restenosis. Known agents, such as FK506, paclitaxel or rapamycin are some examples of drugs capable of being employed to advantage in the context described here.

In particular, the possibility exists of selecting the biodegradation timeframe of the material of the elements 18, in correlation with the time of efficacy of the active principle associated with that material. This in such a fashion that, for example, elements 18 loaded with an active principle of rapid effect degrade more rapidly than elements loaded with an active principle of slower action. It will also be understood that the degradation mechanism and kinetics of the elements 18 may be exploited to control release of the active principle.

According to a particularly advantageous aspect of the solution described here, each of the elements 18 is capable of being made of a different material, or at least of being loaded with a different active principle. This allows the stent to supply different active principles according to their respective release kinetics.

With regard to the manner in which they are coupled to the biodegradable material, different solutions may be employed. The active principle may simply be mixed with the biodegradable material that, gradually becoming consumed, provides gradual release of the active principle.

Above all for those active principles for which rapid delivery is preferred, as an acute dosage, it is also possible to design a co-formation or co-extrusion mechanism. According to this embodiment the elements 18, as initially provided on the stent, are in reality each comprised of two fibers or spaghetti: one consists of biodegradable material and the other of active principle (or of a vector containing active principle), the two fibers being linked together through a co-extrusion mechanism. Co-extrusion techniques of this type are in current use for example in the production of the so-called “conjugated” polyethylene/polypropylene fibers to produce absorbent mass for sanitary articles.

Naturally, if this solution is employed, it is also possible to vary the type and dosage of active principle along the longitudinal extension of the element 18, such as to be able to release, for example, a first active principle (or a larger quantity of a specific active principle) in correspondence with the extremities of the stent and a different type of active principle (or a smaller quantity of the same active principle) at the central portion of the same stent.

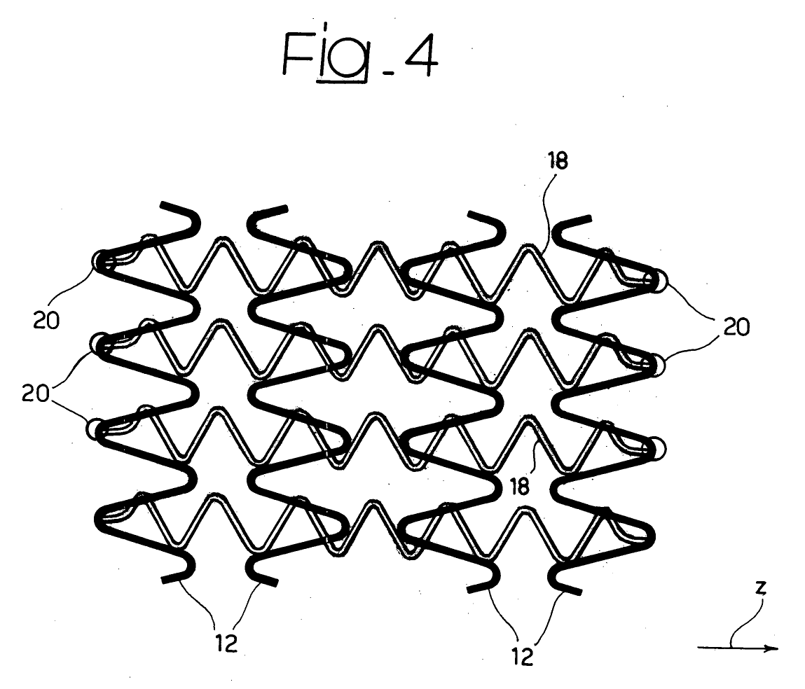

The solution represented in FIG. 4 essentially corresponds to the solution represented in FIG. 3, the difference deriving from the fact that, in this case, instead of presenting a straight line the elements 18 are serpentine, for example following a sinusoidal curve. A solution of this type makes it possible to give the stent great longitudinal flexibility without this being translated into corresponding axial traction stresses with regard to the elements 18. In this case, indeed, the longitudinal flexibility of the stent is chiefly provided by the effect of spreading apart the loops in the trajectory followed by the elements 18.

For the variant represented in FIG. 4 all of the same considerations hold that were made previously with regard, for example, to loading and dosage, as well as the release kinetics of the active principle. Those of skill in the art will however realize that the solution described (in particular the embodiment in which a large number of elements 18 are present, for example approximately 10, distributed around the peripheral outline of the stent, as in the case of the embodiment represented in FIG. 3) makes it possible to apply high dosages of active principle onto the stent. For example, employing the solution represented in FIG. 3, it may be hypothesized that, onto a stent of normal dimensions, quite a large quantity (for example 1 mg) of agent antagonistic to restenosis can be loaded, such as micophenolic acid, rapamycin, tacrolimus, cyclosporin, or corticosteroids.

With regard in particular to the release kinetics of the active principle, it should again be mentioned that elements of an elongated shape such as the elements 18 in FIGS. 3 and 4 lend themselves to acting as vectors for nanoparticles containing an active principle or active principles distributed in a differentiated fashion along the stent. In this connection, an association between fibers of biodegradable material and nanoparticles to which reference may be made is documented in EP-A-1 080 738. In this connection, experts in the sector will immediately realize that, though there may be some affinity between the solutions illustrated, for example, in FIG. 4 of the present application, and the solutions illustrated in FIGS. 1 and 2 of EP-A-1 080 738, an essential conceptual difference exists between the solution described here and the solution described in that previous document of known technique. This fundamental difference lies in the fact that, in the solution documented in EP-A-1 080 738, the fibers containing the nanoparticles are superimposed, combined or in some way interwoven onto a basic structure that of itself is a stent. In particular it continues in all respects to be a stent even if the application of such fibers is not intended.

On the contrary, in the solution described here, the structure of the stent is provided solely by the combination (and by the synergistic cooperation) of the elements 12 and the elements 18. The basic structure represented in FIGS. 1 and 2 of the present application is in fact not of itself capable of providing the full functionality of a stent because it is not of itself able to ensure the features of mechanical coherency and “trackability” necessary to enable implantation of the stent and required in the phases immediately subsequent to implantation.

To ensure this result in the solution described here, the elements 18 must obviously be connected to the elements 12. This comes about in correspondence with anchorage points 20 preferably located in correspondence with the cusps of the loops of the elements 12. This choice derives from the fact that said cusp zones are not subjected to rotation movements during radial expansion of the stent. For the formation of the anchorage points 20 different solutions may be employed (hot welding, cementing) that are compatible with the nature of the material comprising the elements 12 and the material comprising the elements 18. A possible alternative (considered less preferable at present) comprises anchorage by mechanical interlock. This solution may be adopted, for example, when the parts of the cusps of the loops of the elements 18 present an eyelet conformation.

For particular geometric forms of the elements 12 (for example the geometry represented in FIG. 3 of EP-A-0 875 215) it may also be hypothesized that the connection can come about through weaving, in the sense that the elements 18 are woven around the elements 12 and held in position by effect of the weave trajectory.

It will likewise be understood that although in the embodiments represented in FIGS. 3 and 4 the elements 18 extend in a practically continuous fashion along the entire longitudinal extension of the stent, a varied embodiment can undoubtedly be hypothesized in which the elements 18 have a lesser extension, for example linking only adjacent elements 12.

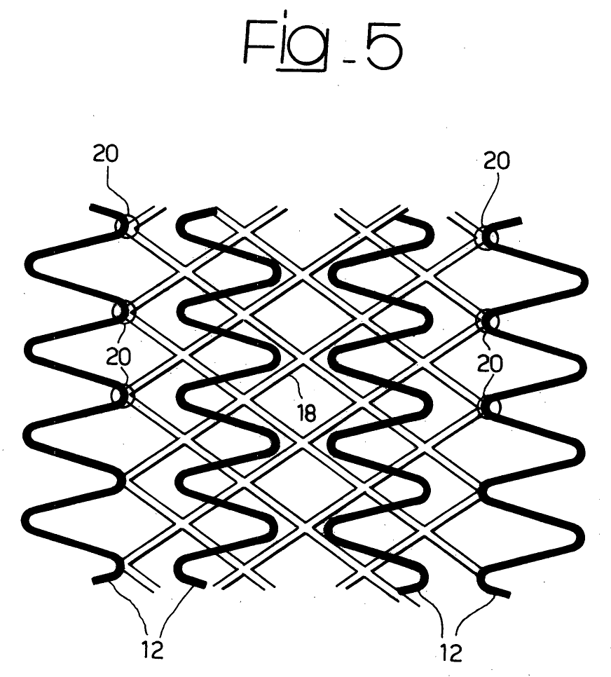

FIG. 5 shows another possible alternative embodiment wherein the elements 18 that extend following a sinusoidal trajectory are not produced as dependent elements but in the form of a network structure of biodegradable material capable of fitting around the basic structure 10 and held there exclusively by elastic forces (although the presence of anchorage or welding points 20, at least in correspondence with the extremities of the stent of the network is undoubtedly to be considered preferable).

Once again, it will not escape the notice of those of skill in the art that, though presenting some similarity with FIG. 6 in EP-A-1 103 234, the solution described here presents an evident and fundamental basic difference compared to the solution described in that previous document of known technique. Once again, in fact, in the solution documented in EP-A-1 103 234 the basic structure onto which the network is applied is of itself a stent.

It will again be appreciated that the application of a layer of biocompatible carbonaceous material following the modalities described, for example, in U.S. Pat. No. 4,624,822, U.S. Pat. No. 4,758,151, U.S. Pat. No. 5,084,151, U.S. Pat. No. 5,133,845, U.S. Pat. No. 5,370,684, U.S. Pat. No. 5,387,247, and U.S. Pat. No. 5,423,886 will preferably involve only the non-biodegradable parts of the stent and will not extend to the biodegradable parts.

Naturally, the principle of the invention holding true, the details of production and the embodiments may be widely varied with regard to what is described and illustrated here, without thereby departing from the sphere of the present invention, as defined by the attached claims. In particular, it will be appreciated that the basic concept of making the tubular structure so that it includes a part of non-biodegradable material and a part of biodegradable material lends itself to embodiments in which some of the annular elements 12 are also made of biodegradable material.

What is claimed is:

1. A stent comprising a tubular structure selectively expandable between a radially-contracted condition and a radially-expanded condition, said tubular structure comprising a part of non-biodegradable material and a part of biodegradable material.

2. A stent according to claim 1, wherein said tubular structure comprises:
a plurality of annular elements aligned along the longitudinal direction of extension of the stent, said annular elements being selectively expandable between a radially-contracted condition and a radially-expanded condition, and
a series of connecting elements that extend in the longitudinal direction of extension of the stent to connect said annular elements, and
wherein said annular elements and said connecting elements are made, respectively, of non-biodegradable material and of biodegradable material.

3. A stent according to claim 2, wherein said annular elements are made of metallic material.

4. A stent according to claim 3, wherein said metallic material is selected from the group consisting of steel and a cobalt-chromium alloy.

5. A stent according to claim 2, wherein said annular elements extend following a looped trajectory.

6. A stent according to claim 2, wherein said annular elements extend following a sinusoidal trajectory.

7. A stent according to claim 6, wherein said plurality of adjacent annular elements extend following a sinusoidal trajectory in phase opposition one to the other.

8. A stent according to claim 2, wherein said plurality of annular elements are connected by longitudinal connecting elements of non-biodegradable material.

9. A stent according to claim 8, wherein said longitudinal connecting elements of non-biodegradable material are substantially non-extensible in the longitudinal direction of the stent.

10. A stent according to claim 8, wherein said longitudinal connecting elements of non-biodegradable material are present, connecting between pairs of said adjacent annular elements, in a lower number than said connecting elements of biodegradable material.

11. A stent according to claim 10, wherein said longitudinal connecting elements of non-biodegradable material, connecting pairs of said annular elements adjacent one to the next, are present to an extent not above 25% of said connecting elements of biodegradable material.

12. A stent according to claim 11, wherein said longitudinal connecting elements of non-biodegradable material, connecting pairs of said annular elements adjacent one to the next, are present to an extent not above 20% of said connecting elements of biodegradable material.

13. A stent according to claim 12, wherein said longitudinal connecting elements of non-biodegradable material, connecting pairs of said annular elements adjacent one to the next, are present to an extent not above 10% of said connecting elements of biodegradable material.

14. A stent according to claim 1, wherein said biodegradable material is a polymer.

15. A stent according to claim 1, wherein said biodegradable material is selected from the group consisting of polylactic acid; poly-ε-caprolactone; polyorthoesters; polyanhydrides; poly-3-hydroxybutyrate; polyaminoacids; polyglycine; polyphosphazenes; polyvinyl alcohol; low molecular weight polyacrylates; and co-polymers of the above.

16. A stent according to claim 1, wherein said biodegradable material is selected from the group consisting of iron and magnesium.

17. A stent according to claim 1, wherein said part of biodegradable material includes connecting elements of biodegradable material extending following a substantially straight trajectory in the longitudinal direction of extension of the stent.

18. A stent according to claim 1, wherein said part of biodegradable material includes connecting elements of biodegradable material that extend following trajectories that are substantially looped in shape, with loops oriented transversally to the longitudinal direction of extension of the stent.

19. A stent according to claim 18, wherein said looped trajectories are sinusoidal trajectories.

20. A stent according to claim 1, wherein said part of biodegradable material includes connecting elements of biodegradable material extending over the entire length of the stent.

21. A stent according to claim 1, wherein said part of biodegradable material includes connecting elements of biodegradable material extending over a part of the length of the stent.

22. A stent according to claim 1, wherein the stent comprises anchorage points between said part of biodegradable material and said part of non-biodegradable material.

23. A stent according to claim 22, wherein said anchorage points are welding spots between said non-biodegradable material and said biodegradable material.

24. A stent according to claim 22, wherein said anchorage points are adhesive points between said non-biodegradable material and said biodegradable material.

25. A stent according to claim 22, wherein said anchorage points are interweaving points between said longitudinal connecting elements of biodegradable material and said annular elements.

26. A stent according to claim 1, wherein said part of biodegradable material forms a network structure that fits over said part of non-biodegradable material.

27. A stent according to claim 1, wherein said part of biodegradable material carries a drug.

28. A stent according to claim 27, wherein said drug is an agent antagonistic to restenosis.

29. A stent according to claim 28, wherein said agent antagonistic to restenosis is selected from the group consisting of paclitaxel, rapamycin, micophenolic acid, rapamycin, tacrolimus, cyclosporin, and corticosteroids.

30. A stent according to claim 27, wherein said part of biodegradable material includes a plurality of connecting elements of biodegradable material that carry different drugs one from the other.

31. A stent according to claim 27, wherein said part of biodegradable material includes a plurality of connecting elements of biodegradable material that carry different dosages of the same drug.

32. A stent according to claim 27, wherein said part of biodegradable material includes at least one connecting element of biodegradable material that carries different drugs along the longitudinal development of the stent.

33. A stent according to claim 27, wherein said part of biodegradable material includes at least one element of biodegradable material that carries different dosages of the same drug along the longitudinal development of the stent.

34. A stent according to claim 27, wherein said drug is mixed with said biodegradable material.

35. A stent according to claim 27, wherein said drug is applied onto said biodegradable material.

36. A stent according to claim 35, wherein said biodegradable material is in the form of fibers and said drug is co-extruded with said fibers.

37. A stent according to claim 27, wherein said drug is disposed on said part of biodegradable material in the form of nanoparticles.

38. A stent according to claim 1, wherein the stent includes a coating of biocompatible carbonaceous material applied on said part of non-biodegradable material.

39. A stent according to claim 38, wherein said part of biodegradable material does not comprise said coating of biocompatible carbonaceous material.
